(19) United States
(12) Patent Application Publication
(10) Pub. No.: US 2010/0204343 A1
(43) Pub. Date: Aug. 12, 2010

(54) 3-(4-HYDROXY-3-METHOXYPHENYL)-1-(4-HYDROXYPHENYL)-1-PROPANONE AND ITS USE AS ANTIOXIDANT

(75) Inventors: Oskar Koch, Gottingen (DE); Gerhard Schmaus, Hoxter-Bosseborn (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/670,522

(22) PCT Filed: Jul. 14, 2008
(86) PCT No.: PCT/EP2008/059183

§ 371 Date: Apr. 10, 2010

Related U.S. Application Data
(60) Provisional application No. 60/951,741, filed on Jul. 25, 2007.

(51) Int. Cl. A61K 47/10; C07C 49/84; A61K 8/35; A23L 3/34

(52) U.S. Cl. 514/772; 568/331; 426/544

(57) ABSTRACT

The present invention relates to 3-(4-hydroxy-3-methoxyphenyl)-1-(4-hydroxyphenyl)-1-propanone of formula (I)

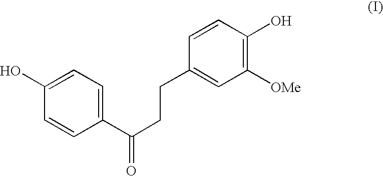

a method for preparation of the compound of formula (I) and its use as an antioxidant in formulations and products.

This invention relates to the use of 3-(4-hydroxy-3-methoxyphenyl)-1-(4-hydroxyphenyl)-1-propanone of formula (I)

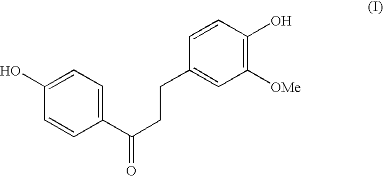

as an antioxidant in cosmetic and/or topical pharmaceutical compositions.

Antioxidants have an impact on chemical compounds that get easily oxidised (oxidative labile chemical compounds) and therefore antioxidants can considerably increase the shelf-life of said compounds in cosmetic and/or topical pharmaceutical compositions.

Therefore natural and synthetic antioxidants find wide use in many different cosmetic and/or topical pharmaceutical products.

In the cosmetic and topical pharmaceutical field referred to a large number of antioxidative active compounds are indeed already employed, but alternatives nevertheless continue to be sought. In this context, however, in the search for alternative agents having an antioxidative activity, it is to be noted that the substances used in the cosmetics and/or topical pharmaceutical field must be
Readily tolerated by the skin,
Toxicologically acceptable,
Stable (in particular in the conventional cosmetic and/or topical pharmaceutical formulations),
Largely and preferably of weak odour or completely odourless,
White or virtually colourless, and
Inexpensive to prepare (i.e. employing standard processes and/or starting from standard precursors).

Besides synthetic antioxidants like butylated hydroxytoluene (BHT), nature also provides very different structural types of antioxidants like carotenoids, anthocyanins, flavonoids, catechins or oligomeric procyanidins possessing strong antioxidant properties. Plant-derived products which are well known to the consumer comprising high concentrations of phenolic antioxidants like catechins or oligomeric proanthocyanidine derivatives (OPC's) are tea leaf extract and grape seed extract, respectively. The antioxidant efficacy of such compounds was proven in numerous in vitro and in vivo studies by many different research groups.

However many of the common antioxidants of synthetic or natural origin are unstable and are coloured per se, resulting in discolouration of cosmetic or pharmaceutical formulations and tend to degrade during the shelf-live of cosmetic and pharmaceutical finished products to inactivederivatives. Furtheron the degradation of such antioxidants is often attributed with discolouration also leading to an undesired browning of finished cosmetic and pharmaceutical formulations.

The aim of the present invention is to provide an alternative antioxidant, which can be advantageously used in cosmetic and/or topical pharmaceutical compositions because of being stable, colourless and keeping its antioxidant activity over a long shelf-life period. Furtheron, degradation to coloured derivatives should also not take place.

The invention also relates to the use of 3-(4-Hydroxy-3-methoxyphenyl)-1-(4-hydroxyphenyl)-1-propanone formula (I)

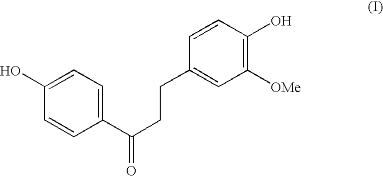

as an antioxidant in cosmetic and/or topical pharmaceutical compositions.

In the sense of this invention topical pharmaceutical compositions mean a pharmaceutical composition which has to be applied topically to skin and/or hair. Preferred topical pharmaceutical compositions are dermatological compositions which have to be applied topically to skin and/or hair.

In the sense of this invention cosmetic compositions are applied also topically to skin and/or hair.

The invention is based on the surprising finding that 3-(4-Hydroxy-3-methoxyphenyl)-1-(4-hydroxyphenyl)-1-propanone of formula (I) has a high activity on scavenging free radicals and is therefore an antioxidative active agent and can be used in cosmetic and/or topical pharmaceutical compositions as the or one of the antioxidants.

3-(4-Hydroxy-3-methoxyphenyl)-1-(4-hydroxyphenyl)-1-propanone is toxicologically acceptable, readily tolerated by the skin, stable in the conventional cosmetic and/or topical pharmaceutical formulations, a white solid in pure form dissolving in carrier systems yielding colourless compositions like cosmetic and/or topical pharmaceutical formulations, largely odourless, showing a slight pleasant odour reminiscent of vanilla beans, and inexpensive to prepare.

3-(4-Hydroxy-3-methoxyphenyl)-1-(4-hydroxyphenyl)-1-propanone of formula (I) can be of natural or of synthetic origin. It is a new chemical substance that has never been described before in literature. Therefore, methods for preparation of 3-(4-hydroxy-3-methoxyphenyl)-1-(4-hydroxyphenyl)-1-propanone, it's antioxidant properties and it's use in cosmetic products, medical devices and pharmaceutical products are also described here for the first time.

Surprisingly, our new research studies showed that 3-(4-hydroxy-3-methoxyphenyl)-1-(4-hydroxyphenyl)-1-propanone of formula (I) possesses excellent radical scavenging activity which are even better to the commonly known antioxidants Trolox (6-hydroxy-2,5,7,8-tetramethylchroman-2-carboxylic acid) or Green Tea extract. Therefore 3-(4-hydroxy-3-methoxyphenyl)-1-(4-hydroxyphenyl)-1-propanone of formula (I) possesses a potent antioxidative activity and can be used as the or one of the antioxidants in cosmetic and/or topical pharmaceutical compositions. Stability studies showed that 3-(4-hydroxy-3-methoxyphenyl)-1-(4-hydroxyphenyl)-1-propanone, in contrast to common antioxidants like Trolox, alpha-tocopherol or green tea extract, is highly stable.

3-(4-Hydroxy-3-methoxyphenyl)-1-(4-hydroxyphenyl)-1-propanone of formula (I) can either be used to protect oxidative labile chemical compounds in cosmetic formulations, medical devices and pharmaceutical products from oxidative degradation and/or deterioration thus prolonging the shelf-life of said formulations.

3-(4-Hydroxy-3-methoxyphenyl)-1-(4-hydroxyphenyl)-1-propanone of formula (I) can also be used in foodstuffs, food products, products for nourishment and beverages.

Preferably 3-(4-hydroxy-3-methoxyphenyl)-1-(4-hydroxyphenyl)-1-propanone is used in a cosmetic and/or topical pharmaceutical composition as described hereinbefore. The topical pharmaceutical composition is preferably a dermatological composition which is to be applied topically.

Preferably the 3-(4-hydroxy-3-methoxyphenyl)-1-(4-hydroxyphenyl)-1-propanone of formula (I) is used in a cosmetic and/or topical pharmaceutical emulsion as described hereinbefore. The topical pharmaceutical emulsion is preferably a dermatological emulsion which is to be applied topically.

The concentration of 3-(4-hydroxy-3-methoxyphenyl)-1-(4-hydroxyphenyl)-1-propanone of formula (I) in cosmetic and/or topical pharmaceutical compositions can range typically from 0.001 to 10 wt.-%, preferably from 0.005 to 5 wt.-% and most preferably from 0.01 to 1.0 wt.-%, based on the total weight of the cosmetic and/or topical pharmaceutical composition.

3-(4-Hydroxy-3-methoxyphenyl)-1-(4-hydroxyphenyl)-1-propanone of formula (I) can be obtained in a manufacturing process comprising the following step:
(b) reduction, preferably hydrogenation, of 3-(4-hydroxy-3-methoxyphenyl)-1-(4-hydroxyphenyl)-2-propen-1-one.

Preferred is a manufacturing process for obtaining the compound of formula (I) comprising the following steps:

(a) reaction (condensation) of 4-hydroxy acetophenone and vanillin,
(b) reduction, preferably hydrogenation, of 3-(4-hydroxy-3-methoxyphenyl)-1-(4-hydroxyphenyl)-2-propen-1-one obtained in step (a).

3-(4-Hydroxy-3-methoxyphenyl)-1-(4-hydroxyphenyl)-2-propen-1-one is a known compound and is described for example in DE 1 099 732 (for manufacturing polycarbonates), DE 1 447 016 (for manufacturing light sensitive comaterials), DE 2 256 961 (for manufacturing expoy resins), Chem. Pharm. Bull. 1983, 31(1), 149-155 (investigation of inhibiting phenylalanin-ammonia-lyase), Designed Monomers and Polymers (2003), 6(2), 187-196, High Performance Polymers (2006), 18(2), 227-240 and J. of Macromolecular Science, Part A: Pure and Applied Chemistry (2005), A42(12), 1589-1602 (manufacturing of polymers), Journal of Natural Products (2006), 69(2), 191-195 (detection in Allium atroviolaceum).

The invention is explained in more detail in the following examples, whereby the present invention is not limited to the disclosed examples.

### EXAMPLE 1

### Synthesis of 3-(4-hydroxy-3-methoxyphenyl)-1-(4-hydroxyphenyl)-1-propanone of formula (I)

20 g sodium hydroxide are diluted in 100 g diethyleneglycol diethylether and heated up to 120° C. under stirring. A mixture of 14 g p-hydroxy acetophenone and 15 g of vanillin are continuously added over a period of 1 hour. After stirring for further 20 min and hydrolysis the pH is adjusted between 6-7.25 g of crystalline 3-(4-hydroxy-3-methoxyphenyl)-1-(4-hydroxyphenyl)-prop-2-en-1-one are obtained after phase separation and evaporation of the solvent. Yield: 93% of theory.

10 g 3-(4-hydroxy-3-methoxyphenyl)-1-(4-hydroxyphenyl)-prop-2-en-1-one are diluted in 100 g tetrahydrofuran (THF) and 0.2 g Pd on charcoal (Pd/C; Pd-content: 5 wt.-%, water-content: 50 wt.-%, each based on the total weight of the catalyst) are added. After hydrogenation of 3-(4-Hydroxy-3-methoxyphenyl)-1-(4-hydroxyphenyl)-prop-2-en-1-one at normal pressure and ambient temperature (approx. 20° C.) 9 g of 3-(4-hydroxy-3-methoxyphenyl)-1-(4-hydroxyphenyl)-1-propanone of formula (I) are obtained after separation from the catalyst and evaporation of the solvent. Yield: 89% of theory.

Spectroscopic data of the compound of formula (I): ¹³C-NMR (CDCl₃; 75.5 MHz): 8 (ppm)=197.42 (s), 161.85 (s), 147.24 (s), 144.44 (s), 132.03 (s), 130.37 (d), 130.37 (d), 128.18 (s), 120.27 (d), 115.11 (d), 115.07 (d), 115.07 (d), 112.53 (d), 55.42 (q), 39.30 (t), 29.42 (t); MS: m/z (%)=M⁺-Ion 272(82), 151(24), 137(77), 121(100), 93(19), 65(22).

### EXAMPLE 2

### ABTS Assay—Measurement of the Antioxidant Capacity of 3-(4-hydroxy-3-methoxyphenyl)-1-(4-hydroxyphenyl)-1-propanone of formula (I)

The antioxidant capacity of 3-(4-hydroxy-3-methoxyphenyl)-1-(4-hydroxyphenyl)-1-propanone of formula (I) disclosed here was evaluated with the ABTS assay, a cell free in vitro test. The assay principle bases on the chemical reduction being accompanied by discolouration of the green coloured cationic radical 2,2′-Azinobis(3-ethylbenzothiazoline 6-sulfonic acid) (ABTS⁺) by antioxidants. The grade of discolouration can be measured photometrically at 734 nm. The compound of formula (I) as well as standard antioxidants like Trolox, a water soluble alpha-tocopherol analog and green tea extract were tested on 96-well microtiter plates at 5 concentrations. Each concentration was tested in double in two independent experiments. IC₅₀ values for the compound of formula (I) as well as for the comparative examples are depicted in table 1.

The ABTS study for 3-(4-hydroxy-3-methoxyphenyl)-1-(4-hydroxyphenyl)-1-propanone of formula (I) exemplifies that this new chemical possesses a very high antioxidant capacity showing even better results than several well known products like Trolox or green tea extract. Therefore, the compound of formula (I) is a highly suitable new product either to protect biological systems from damage caused by free radicals of different type and origin or to extend the shelf life of products comprising highly unstable, easily oxidising molecules.

TABLE 1
IC₅₀ values for 3-(4-hydroxy-3-methoxyphenyl)-1-
(4-hydroxyphenyl)-1-propanone of formula (I),
Trolox (2H-1-Benzopyran-2-carboxylic acid, 3,4-
dihydro--hydroxy-2,5,7,8-tetramethyl-;
6-hydroxy-,5,7,8-etramethylchroman-2-
carboxylic acid) and green tea extract measured
with the BTS method:

| Substance | IC₅₀ [mg/ml] |
|---|---|
| 3-(4-hydroxy-3-methoxyphenyl)-1-(4- | 0.005 |
| hydroxyphenyl)-1-propanone of | |
| formula (I) | |
| Trolox | 0.006 |
| Green Tea Extract | 0.007 |

1. A compound of formula (I)

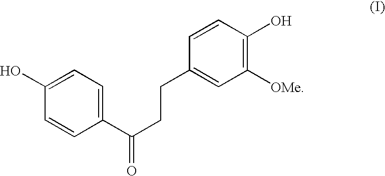

2. A method of protecting an oxidative labile chemical from oxidation comprising combining an antioxidative active amount of a compound of Formula (I), 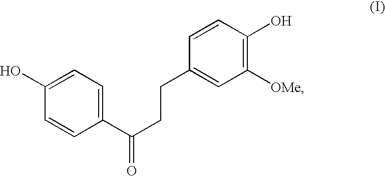
with the oxidative labile chemical.

3. The method according to claim 2, wherein the compound of formula (I) is used to protect an oxidative labile chemical in cosmetic and/or topical pharmaceutical compositions and to prolong the shelf-life of cosmetic formulations, medical devices and pharmaceutical products.

4. A composition comprising:
(a) an antioxidative active amount of the compound of formula (I), 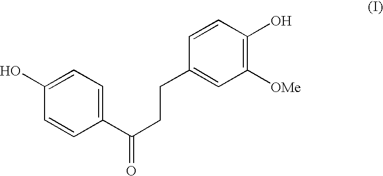
and
(b) a carrier system that is compatible with the compound of formula (I).

5. The composition according to claim 4, wherein said composition is selected from the group consisting of cosmetic compositions, medical devices, pharmaceutical compositions, foodstuffs, food products, products for nourishment and beverages.

6. The composition of claim 4 comprising from 0.001 to 10 wt % of the compound of formula (I) based on the total weight of the composition.

7. The composition of claim 6 comprising from 0.005 to 5 wt % of the compound of formula (I) based on the total weight of the composition.

8. The composition of claim 7 comprising from 0.01 to 1.0 wt % of the compound of formula (I) based on the total weight of the composition.

9. A method of extending the shelf-life of a composition comprising adding a compound of formula (I) 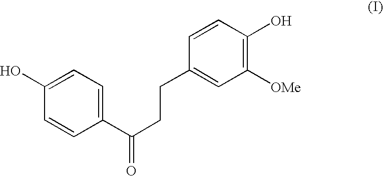
to the composition and extending the shelf-life of the composition.

10. The method of claim 9, wherein the composition is a cosmetic composition.

11. The method of claim 9, wherein the composition is a pharmaceutical composition.

12. The method of claim 9, wherein the pharmaceutical compositions is a topical pharmaceutical composition.

13. The method of claim 9, wherein the composition is a medical device.

14. The method of claim 9, wherein the composition is a food product.

15. The method of claim 9, wherein the composition is a beverage.

16. A method of scavenging free radicals comprising bringing the free radicals into contact with a compound of formula (I) 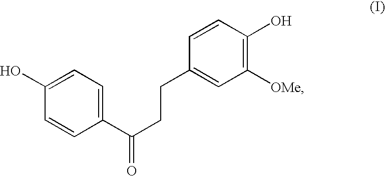
and oxidizing the compound of formula (I).
